(19) United States
(12) Patent Application Publication
(10) Pub. No.: US 2021/0322450 A1
(43) Pub. Date: Oct. 21, 2021

(54) APPLICATION OF COMPOSITION CONTAINING NICOTINAMIDE MONONUCLEOTIDE IN ANTI-AGING DRUGS/HEALTHCARE PRODUCTS

(71) Applicant: HOBOOMLIFE BIO-TECHNOLOGY (SHENZHEN) CO., LTD., Shenzhen (CN)

(72) Inventors: Jiansheng Chen, Shenzhen (CN); Zhigang Duan, Shenzhen (CN)

(21) Appl. No.: 17/366,022

(22) Filed: Jul. 1, 2021

Related U.S. Application Data
(63) Continuation of application No. PCT/CN2020/075619, filed on Feb. 18, 2020.

(30) Foreign Application Priority Data
Jun. 6, 2019 (CN) 201910503708.1

(51) Int. Cl. A61K 31/706; A61K 31/519; A61K 31/05; A61K 31/352; A61K 31/12; A61K 31/7048; A61P 39/00

(52) U.S. Cl. CPC A61K 31/706; A61K 31/519; A61K 31/05; A61P 39/00; A61K 31/12; A61K 31/7048; A61K 31/352

(57) ABSTRACT

The application of a composition containing nicotinamide mononucleotide in anti-aging drugs/healthcare products. Also provided is a preparation method for said composition. NMN can have the function of activating the energy metabolism of an organism and improving the oxidative stress response of the organism and, in synergy with other components, has a good anti-aging effect; each component in the composition is structurally stable, and will not easily deteriorate or be damaged after preparation of a corresponding product; and each component is safe and has no substantial adverse reaction with the human body; the technical problem in the prior art of the difficulty for anti-aging products to have good anti-aging effects whilst being harmless to the human body and having stable product quality is thus solved.

### CROSS REFERENCE TO RELATED APPLICATIONS

The present application is a Continuation Application of PCT Application No. PCT/CN2020/075619 filed on Feb. 18, 2020, which claims the benefit of Chinese Patent Application No. 201910503708.1 filed on Jun. 6, 2019. All the above are hereby incorporated by reference in their entirety.

### FIELD OF THE INVENTION

The present application belongs to the field of research and development of drugs and healthcare products, and particularly relates to an application of a composition containing nicotinamide mononucleotide in anti-aging drugs/healthcare products.

### BACKGROUND OF THE INVENTION

With the continuous improvement of living standards, the desire for people to pursue healthy and longevity is becoming more and more intense. Aging, however, is an inevitable regular rule in any life process, and is a biological rule that is not intended to be transferred by a person. But it is possible to prolong the lifespan of a person to reach his highest life which is naturally imparted by retarding the aging speed. To date, there are hundreds of doctrines and assumptions regarding aging mechanisms, such as free radical theory, immune function degradation theory, neuroendocrine theory, protein synthesis error accumulation theory, and the like, as well as gene regulation theory, DNA damage repair theory, mitochondrial injury theory, and telomerase theory, which are based on molecular level and gene level in recent years.

### SUMMARY OF THE INVENTION

### Technical Problems

In the prior art, although the anti-aging mechanism has a wide variety of research, there are few anti-aging products. Most of the anti-aging products on the market are a combination of the extracts of food or Chinese medicine. To some extent, they can enhance the function of the human body, but the effect is not ideal and the anti-aging effect needs to be promoted. Industrial ingredients with strong antioxidant activity are used in certain anti-aging products to achieve good anti-aging effects, which results in quick response, but it is harmful to human body when being used continuously in a long time. Vitamin antioxidants are added into some other anti-aging products, but this kind of products have poor stability.

Therefore, it has become an urgent problem for those skilled in the art to study the application of a composition containing nicotinamide mononucleotide in anti-aging drugs/healthcare products, which can be used to solve the technical problem in the prior art for anti-aging products to have good anti-aging effects whilst being harmless to the human body and having stable product quality.

### TECHNICAL SOLUTION OF THE INVENTION

### Technical Solutions

In view of the above, the present application provides an application of a composition containing nicotinamide mononucleotide in anti-aging drugs/healthcare products, which is used for solving the technical problem in the prior art for anti-aging products to have good anti-aging effects whilst being harmless to the human body and having stable product quality.

The present invention provides a composition, the composition comprises the following raw materials: nicotinamide mononucleotide (NMN), rutecarpine, resveratrol, fisetin, butein, icariin, and honokiol.

Preferably, the composition comprises the following raw materials in parts by mass: 1-10 parts of nicotinamide mononucleotide, 1-10 parts of rutecarpine, 1-10 parts of resveratrol, 1-10 parts of fisetin, 1-10 parts of butein, 1-10 parts of icariin and 1-10 parts of honokiol.

Preferably, the composition comprises the following raw materials in parts by mass: 2-8 parts of nicotinamide mononucleotide, 5-7 parts of rutecarpine, 2-5 parts of resveratrol, 3-6 parts of fisetin, 1-5 parts of butein, 6-9 parts of icariin and 2-5 parts of honokiol.

Preferably, a dosage form of the composition is an oral formulation and/or a parenteral administration dosage form.

The oral formulation is selected from any one or more of a tablet, a powder, a capsule, a granule, a pill, a suspension, a syrup, a mixture, a pulvis, and a drop pill; the parenteral administration dosage form is selected from any one or more of an injection, an inhalant, a patch, a suppository, and an ointment.

The present application also provides a method for preparing any one of the above-mentioned compositions, wherein the method comprises the following steps:

Step 1, drying and sieving nicotinamide mononucleotide, rutecarpine, resveratrol, fisetin, butein, icariin and honokiol respectively, and then mixing the resulting materials uniformly to obtain a first product;

Step 2, drying auxiliary materials and mixing uniformly, and then sieving the resulting materials to obtain a second product;

Step 3, dissolving the first product and the second product in water to obtain a mixture and stiffing the mixture for a first time, then heating the mixture and stiffing the mixture for a second time, preparing the resulting materials into a dosage form to obtain the product.

Preferably, in step 1, a particle size of the sieve is 60-80 meshes; in step 2, a particle size of the sieve is 60-80 meshes.

Preferably, in step 2, the auxiliary material is selected from any one or more of mannitol, microcrystalline cellulose, magnesium stearate, carboxymethyl cellulose, and calcium hydrophosphate.

Preferably, in step 3, a temperature of the first stiffing is 25-40° C., and a temperature of the second stiffing is 50° C., and a time for the second stiffing is 1 hour.

The invention also provides an application of any one of the above-mentioned compositions or the product obtained by any one of the above-mentioned methods in anti-aging drugs and/or healthcare products.

### BENEFICIAL EFFECTS OF THE INVENTION

### Beneficial Effects

In summary, the present application provides a composition comprising the following raw materials: nicotinamide mononucleotide, rutecarpine, resveratrol, fisetin, butein, icariin and honokiol. The invention also provides a method for preparing the composition. The invention also provides an application of the above-mentioned composition or the product obtained by the above-mentioned method in anti-aging drugs and/or healthcare products. In the technical solution provided by the invention, the nicotinamide mononucleotide can have the function of activating the energy metabolism of an organism and improving the oxidative stress response of the organism, and meanwhile, in synergy with the rutecarpine, the resveratrol, the fisetin, the butein, the icariin and the honokiol, has a good anti-aging effect. And each component in the composition is structurally stable, and will not easily deteriorate and be damaged after preparation of a corresponding product; meanwhile, each component is safe, and has no substantial adverse reactions with the human body. The invention of the composition containing nicotinamide mononucleotide provided by the invention in anti-aging drugs/healthcare products solves the technical problem in the prior art for anti-aging products to have good anti-aging effects whilst being harmless to the human body and having stable product quality.

### BRIEF DESCRIPTION OF THE DRAWINGS

In order to more clearly illustrate the embodiments of the present application or the technical solutions in the prior art, the following will briefly introduce the drawings to be used in the description of the embodiments or the prior art. Obviously, the drawings in the following description are only some embodiments' drawings of the present application. For those of ordinary skill in the art, other drawings may also be obtained based on these drawings without creative work.

wherein:

FIG. 1 shows the results of SIRT1 expression in the aorta of young and old controls (YC and OC) mice (n=7 per group), as well as young and old mice (n=7 per group) of the group of the composition of the present application (Y—the young experimental group of the present application, O—the old experimental group of the present application).

FIG. 2 shows the results of the ratio of acetylated to total NFκB in the aorta of young and old controls (YC and OC) mice (n=7 per group), as well as young and old mice (n=7 per group) of the group of the compositions of the present application (Y—the young experimental group of the present application, O—the old experimental group of the present application).

FIG. 3 shows the results of dose response to endothelial-dependent dilator acetylcholine (ACh) of young and old controls (YC and OC) mice (n=10 per group), as well as young and old mice (n=10 per group) of the group of the compositions of the present application (Y—the young experimental group of the present application, O—the old experimental group of the present application).

FIG. 4 shows the results of the maximum reaction dose of the endothelium-dependent dilator acetylcholine (ACh) in young and old controls (YC and OC) mice (n=10 per group), as well as young and old mice (n=10 per group) of the group of the compositions of the present application (Y—the young experimental group of the present application, O—the old experimental group of the present application) in the presence or absence of TEMPOL.

FIG. 5 shows the results of superoxide generation evaluated by electron paramagnetic resonance (EPR).

FIG. 6 shows the results of the aortic pulse wave velocity ((a PWV) of young and old control (YC and OC) mice (n=10 per group), as well as youth and old mice (n=10 per group) of the group of the compositions of the present application (Y—the young experimental group of the present application, O—the old experimental group of the present application).

FIG. 7 shows the results of the elastic modulus (N=10/group) of young and old control (YC and OC) mice (n=10 per group), as well as young and old mice (n=10 per group) of the group of the compositions of the present application (Y—the young experimental group of the present application, O—the old experimental group of the present application).

FIG. 8 shows the results of the total elastin expression (N=10/group) of young and old control (YC and OC) mice (n=10 per group), as well as young and old mice (n=10 per group) of the group of the compositions of the present application (Y—the young experimental group of the present application, O—the old experimental group of the present application).

### EMBODIMENTS OF THE INVENTION

The technical solutions in the embodiments of the present application will be clearly and completely described below in conjunction with the drawings in the embodiments of the present application, and obviously, the described embodiments are merely a part of the embodiments of the present application, rather than all embodiments. Based on the embodiments in the present application, all other embodiments obtained by those of ordinary skill in the art without creative work shall fall within the protection scope of the present application.

The present application provides an application of a composition containing nicotinamide mononucleotide in anti-aging drugs/healthcare products, which can be used to solve the technical problem in the prior art for anti-aging products to have good anti-aging effects whilst being harmless to the human body and having stable product quality.

The technical solutions in the embodiments of the present application will be described clearly and completely below. Obviously, the described embodiments are merely a part of the embodiments of the present application, rather than all the embodiments. Based on the embodiments of the present application, all other embodiments obtained by those of ordinary skill in the art without creative work shall fall within the protection scope of the present application.

For a more detailed description of the present application, the application of the composition containing nicotinamide mononucleotide in anti-aging drugs/healthcare products provided by the present application will be specifically described below in conjunction with embodiments.

The present application provides a composition comprising: nicotinamide mononucleotide (NMN), rutecarpine, resveratrol, fisetin, butein, icariin, and honokiol. The application of the composition containing nicotinamide mononucleotide provided by the present application in anti-aging drugs/healthcare products solves the technical problem in the prior art for anti-aging products to have good anti-aging effects whilst being harmless to the human body and having stable product quality.

Sirtuins protein family is widely distributed in cells, and participates in the regulation of cell differentiation and apoptosis, cell cycle, metabolism, genome stability, and the like. NMN is the precursor of NAD+. After entering the cell, it becomes NAD+, an important coenzyme involved in many reactions in human body. The NAD+ dependent histone deacetylases Sirtuins family can promote mitochondrial autophagy and regeneration.

Mammalian SIRT1 is one of the seven members of the protein deacetylase/deacetylase sirtuin family. It is a nicotinamide adenine dinucleotide (NAD+) dependent deacetylase, a sensor of energy metabolism of the body and improvemen oxidative stress of the body. Studies have shown that decreased expression and increased activity of SIRT1 in aging arteries is a key mechanism for regulating damaged EDD (endothelial-dependent expansion).

In the technical solutions provided by the present application, by supplementing NAD+ key intermediate nicotinamide mononucleotide (NMN), SIRT1 can be activated and the metabolism and stress response of elderly mice can be improved. NMN supplementation can increase the activity of arterial SIRT1, reduce vascular oxidative stress, and reverse vascular dysfunction caused by aging. At the same time, NMN also reduces the production of vascular superoxide, increases the bioavailability of NO (nitric oxide), and affects the expression of collagen and elastin.

At the same time, the resveratrol in the composition can directly or indirectly activate SIRT1 to play an antioxidant role or activate SIRT1 gene, which can effectively prevent or delay the occurrence and development of many age-related diseases.

Further, the icariin in the composition can up-regulate the expression of SIRT1-dependent antioxidant enzymes, thereby reducing the oxidative stress produced by ROS; the honokiol in the composition can activate a key protective protein SIRT3, which is closely related to anti-aging, stress resistance and metabolic regulation; and the resveratrol, the fistein, the butein and the like in the composition have strong antioxidant and anti-free radical effects, mainly by activating the activity of histone deacetylase (Sirtuin, silent mating type information regulation 2 homolog), and then regulating the transcription and activity of downstream genes; and the rutecarpine in the composition can inhibit endothelial cell aging induced by high glucose, and its mechanism is related to activating the TRPV1/[Ca˜−(2+)]i signal pathway, up-regulating the longevity protein SIRT1, and then down-regulating the aging-related protein P21, and inhibiting the generation of ROS.

Therefore, the rutecarpine, resveratrol, honokiol, fisetin, butein, icariin and NMN have a synergistic effect to increase the activation of sirtuins protein by NMN, thereby changing the activity and stability of the protein, so as to play a role in regulating the aging process.

In the technical solution provided by the present application, specific ingredients and specific ratios are adopted, the formula is reasonable, the compatibility is appropriate. The technical solution conforms to the theory of combining traditional Chinese medicine and modern medicine, is convenient to use, has good absorption effect, and has no adverse effects and side effects. Through clinical verification, it can effectively reduce free radicals in the body and maintain the level of active oxygen, thereby improving cell damage.

### Example 1

This present example is a specific example of preparing product 1.

Nicotinamide mononucleotide, rutecarpine, resveratrol, fisetin, butein, icariin and honokiol were dried and passed through a 60-80 mesh sieve, and mixed uniformly to obtain a first product.

After being dried, the auxiliary materials were mixed uniformly and passed through a 60-80 mesh sieve to obtain a second product. In this example, the auxiliary material was mannitol.

After the first product and the second product were dissolved in water, they are stirred for a first time at 25-40° C., stirred evenly and then heated up, and stirred for a second time at 50° C., and after being stirred for 1 hour, prepared the resulting materials into a dosage form to obtain the tablet product 1; in this example, the formulation process used was a conventional formulation process well known to those skilled in the art, and will not be further described herein.

In this example, in the first product, the amount of each raw material is 10 g of nicotinamide mononucleotide, 5 g of rutecarpine, 10 g of resveratrol, 6 g of fisetin, 5 g of butein, 9 g of icariin and 2 g of honokiol.

### Example 2

This present example is a specific example of preparing product 2.

Nicotinamide mononucleotide, rutecarpine, resveratrol, fisetin, butein, icariin and honokiol were dried and passed through a 60-80 mesh sieve, and mixed uniformly to obtain a first product.

After being dried, the auxiliary materials were mixed uniformly and passed through a 60-80 mesh sieve to obtain a second product. In this example, the auxiliary material was microcrystalline cellulose.

After the first product and the second product were dissolved in water, they are stirred for a first time at 25-40° C., stirred evenly and then heated up, and stirred for a second time at 50° C., and after being stirred for 1 hour, prepared the resulting materials into a dosage form to obtain the pill product 2. In this example, the formulation process used was a conventional formulation process well known to those skilled in the art, and will not be further described herein.

In this example, in the first product, the amount of each raw material is 5 g of nicotinamide mononucleotide, 10 g of rutecarpine, 1 g of resveratrol, 5 g of fisetin, 10 g of butein, 7 g of icariin, and 1 g of honokiol.

### Example 3

This present example is a specific example of preparing product 3.

Nicotinamide mononucleotide, rutecarpine, resveratrol, fisetin, butein, icariin and honokiol were dried and passed through a 60-80 mesh sieve, and mixed uniformly to obtain a first product.

After being dried, the auxiliary materials were mixed uniformly and passed through a 60-80 mesh sieve to obtain a second product. In the example, the auxiliary material was magnesium stearate.

After the first product and the second product were dissolved in water, they are stirred for a first time at 25-40° C., stirred evenly and then heated up, and stirred for a second time at 50° C., and after being stirred for 1 hour, prepared the resulting materials into a dosage form to obtain the granule product 3. In this example, the formulation process used was a conventional formulation process well known to those skilled in the art, and will not be further described herein.

In this example, in the first product, the amount of each raw material is 2 g of nicotinamide mononucleotide, 7 g of rutecarpine, 2 g of resveratrol, 10 g of fisetin, 1 g of butein, 10 g of icariin and 5 g of honokiol.

### Example 4

This present example is a specific example of preparing product 4.

Nicotinamide mononucleotide, rutecarpine, resveratrol, fisetin, butein, icariin and honokiol were dried and passed through a 60-80 mesh sieve, and mixed uniformly to obtain a first product.

After being dried, the auxiliary materials were mixed uniformly and passed through a 60-80 mesh sieve to obtain a second product. In this example, the auxiliary material was carboxymethyl cellulose.

After the first product and the second product were dissolved in water, they are stirred for a first time at 25-40° C., stirred evenly and then heated up, and stirred for a second time at 50° C., and after being stirred for 1 hour, prepared the resulting materials into a dosage form to obtain the capsule product 4; in this example, the formulation process used was a conventional formulation process well known to those skilled in the art, and will not be further described herein.

In this example, in the first product, the amount of each raw material is 1 g of nicotinamide mononucleotide, 6 g of rutecarpine, 5 g of resveratrol, 1 g of fisetin, 2 g of butein, 6 g of icariin, and 10 g of honokiol.

### Example 5

This present example is a specific example of preparing product 5.

Nicotinamide mononucleotide, rutecarpine, resveratrol, fisetin, butein, icariin and honokiol were dried and passed through a 60-80 mesh sieve, and mixed uniformly to obtain a first product.

After being dried, the auxiliary materials were mixed uniformly and passed through a 60-80 mesh sieve to obtain a second product. In this example, the auxiliary material was calcium hydrophosphate.

After the first product and the second product were dissolved in water, they are stirred for a first time at 25-40° C., stirred evenly and then heated up, and stirred for a second time at 50° C., and after being stirred for 1 hour, prepared the resulting materials into a dosage form to obtain the water agent product 5; in this example, the formulation process used was a conventional formulation process well known to those skilled in the art, and will not be further described herein.

In this example, in the first product, the amount of each raw material is 8 g of nicotinamide mononucleotide, 1 g of rutecarpine, 3 g of resveratrol, 3 g of fisetin, 3 g of butein, 1 g of icariin and 4 g of honokiol.

### Example 6

### 6.1 Animal Treatment

Young (3-4 months) and old (20-24 months) C57BL/6 male mice were purchased. All mice were bred in an animal house for 2 weeks, and the light was adjusted according to the rhythm of 13 hours of daylight. During the breeding period, the mice can freely intake water and food, the feeding environment temperature was always kept at 20° C.-22° C., and the relative humidity was 50%-60%.

After a two-week adaptation period, the young and old mice were divided into two subgroups: control group animals (YC—young control group, OC—old control group) continued to drink normal water, and other groups of animals (Y—the youth experimental group of the present application, O—the old experimental group of the present application) received drinking water contained product 1 prepared by the present application (target dose of 300 mg/kg/day) for 8 weeks, their body weight and water intake were monitored 3 times a week.

### 6.2.1 Ex-Vivo Carotid Artery Vasodilation Reactions

After 8 weeks, the mice were anesthetized with isoflurane and euthanized by bloodletting through cardiac puncture. The carotid artery was excised, the vein was inserted into the tip of a glass micropipette, and fixed with a nylon suture in a myograph chamber (DMTInc., Ann Arbor, Mich., USA) containing a buffered saline solution.

The artery was pressurized to 50 mm Hg at 37° C. and equilibrated for 45 minutes before the experiment. After sub-polar contraction was performed using phenylephrine (2 μM), the diameter of the arterial lumen was increased to the degree of response of acetylcholine (Ach: 1×10⁻⁹1×10⁻⁴ M), and the arterial tube treated with acetylcholine was used to determine endothelial-dependent dilation by vasodilation in response to NO donor sodium nitroprusside (SNP: 1×10⁻¹⁰1×10⁻⁴ M). Calculated according to the following formula, the vasodilation response was recorded as the actual diameter due to the difference in the maximum carotid artery diameter of the young and old animals, expressed as a percentage of the maximum reaction. The formula is as follows:

Dilation (%)=(Ds−Db)/(Dm−Db)×100%;

wherein, Dm is the maximum diameter of the lumen under a pressure of 50 mm Hg pressure; Db is the steady-state lumen diameter after pre-contraction before the first addition of the drug; Ds is the steady-state lumen diameter recorded after the drug is added.

### 6.2.2 Body Aortic Pulse Wave Velocity

The aortic pulse wave velocity (aPWV) was measured, the mice were anesthetized with 2% isoflurane and lied on its back, and the both legs were fixed on an electrocardiogram (ECG) electrode. The aortic blood flow velocity in the transverse aortic arch and the abdominal aorta was measured with a Doppler probe. For each site, the time before ejection, that is, the time between the R-wave of the ECG and the bottom of the Doppler signal was determined. The aPWV was calculated by dividing the distance between the lateral probe and the abdominal probe by the difference in the pre-injection time between the chest and the abdominal.

### 6.2.3 Measurement of Superoxide in Aorta

The aortic superoxide was measured using an EPR spectroscopy. The 1 millimeter aortic segment without perivascular fat and other surrounding tissues was incubated with the superoxide specific rotating probe 1-hydroxy-3-methoxycarbonyl-2,2,5,5-tetramethylpyrrolidine in Krebs-Hepes buffer for one hour at 37° C. to detect amount of superoxide produced by whole cell. The signal amplitude was analyzed using an MS 300X band EPR spectrometer. The specific settings were as follows: Midfield, 3350 G; Scan, 80 G, Microwave Modulation, 3000 mG, and Microwave Attenuation 7 dB.

### 6.2.4 Measurement of NAD+ Content in Mice

The aorta NAD+ level was measured by an HPLC system with a Supelco LC-18-T columns (15 cm×4.6 cm) in young (3-4 months) mice.

### 6.3 Experimental Results

### 6.3.1 the NMN Composition Activates Arterial SIRT1

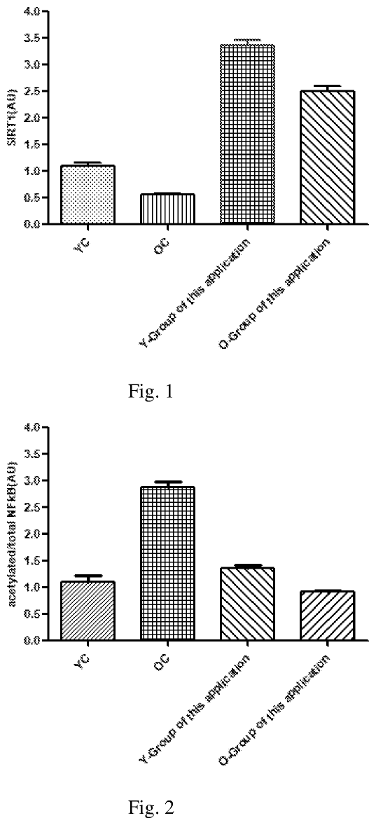

Compared with young mice, the average expression level of SIRT1 in the aorta of the old mouse was about 40% (FIG. 1). The expression level of SIRT1 protein was increased in young mice and old mice after supplementing with the compositions of the present application, and the expression level of SIRT1 in young mice was increased greater than young mice (FIG. 2).

The p65 subunit of NFκB is the main target of SIRT1, which is the response of deacetylation to the activity of SIRT1. Thus, SIRT1 activation was determined by evaluating the ratio of acetylation to total NFκB (p65 subunit). Compared with the young controls, the ratio in the aorta of the old control group was higher, indicating that the aortic SIRT1 activity decreases with aging. Most importantly, the compositions of the present application restore the activity of SIRT1 in the aorta of the old group.

### 6.3.2 Restore the Maximum EDD of Acetylcholine Dependence in Old Mice

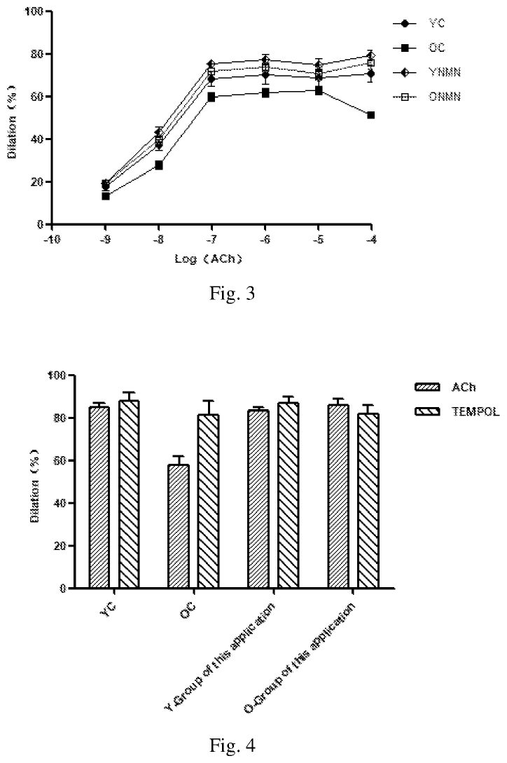

In vitro evaluation of the maximum degree EDD of dependent acetylcholine in the young group and the old group of mice, the old group was significantly lower than the young group. The composition of the invention obviously alleviated the maximum degree EDD of dependent acetylcholine in the old group of mice, but had no obvious effect on the young group of mice, (as shown in FIG. 3).

### 6.3.3 NMN Reduces Vascular Oxidative Stress

The ex-vivo carotid artery segment of the mouse was incubated with superoxide dismutase analog 4-hydroxy-2,2,6,6-tetramethylpiperidin-1-yloxy (TEMPOL) in vitro, which restored the EDD of the carotid artery in the old control group but had no effect on other groups (FIG. 4). This indicates that excess superoxide mediates endothelial dysfunction with age.

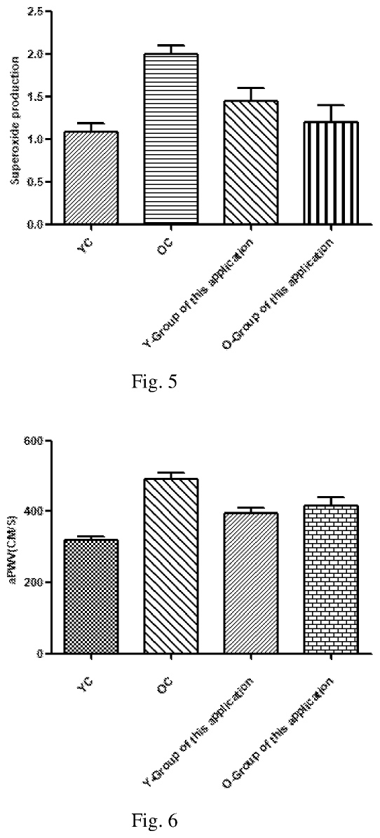

In order to further evaluate the effects of aging and treatment with the composition of the present application on arterial oxidative stress, the aorta was used for relevant biochemical analysis. Direct evaluation by electron paramagnetic resonance (EPR) spectroscopy showed that the superoxide content in the aorta of the old group of mice was significantly higher than that of the young group (FIG. 5), which was consistent with the pharmacological function test results of TEMPOL.

### 6.4.5 the Stiffness of the Aorta in Mice is Normalized

The large elastic arteriosclerosis evaluated by aPWV in vivo was significantly higher in the old control group than in the young control group (FIG. 6). Supplementing the composition of the present application reversed the age-related increase in aPWV in old mice, but had no significant effect on young mice (FIG. 6).

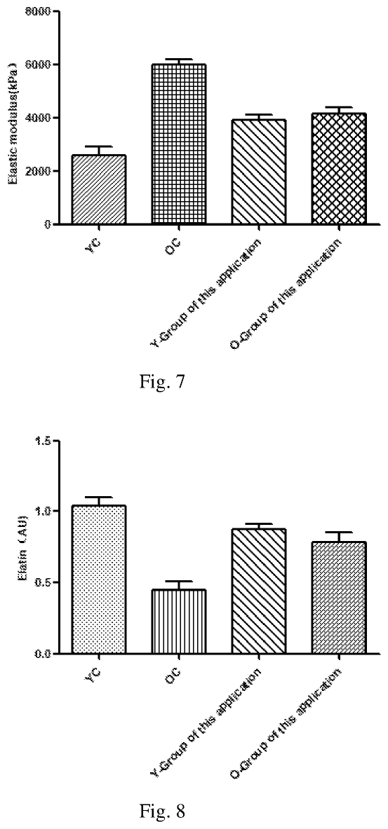

Similarly, the in vitro arteriosclerosis index of the old group was significantly higher than that of the young group. After supplementing the composition of the present application, this value returned to normal (FIG. 7). The content of elastic collagen in the old group was significantly lower than that of the young group (FIG. 8), after supplementing the composition of the present invention, the content of elastic collagen in the old group tended to be the normal group.

The above results show that the compositions of the present application reversed the growth of large elastic arteriosclerosis with age, in part by retaining elastin in the arterial wall to achieve this function.

Repeat the above examples for products 2-5, and similar experimental results were obtained, which will not be repeated here.

It can be concluded from the foregoing examples that oral supplementation of the product obtained from the technical solution of the present application increases the expression of mouse SIRT1 protein, and SIRT1 promotes self-transcription regulation through enhanced deacetylation and transcription factor activity. As the body's longevity protein, SIRT1 participates in the body's multi-purpose physiological processes. It delays the body's aging and maintains the body's health by protecting cells from oxidative stress, protecting nerves, and promoting the production of bone and muscle.

In summary, the present application provides a composition comprising the following raw materials: nicotinamide mononucleotide, rutecarpine, resveratrol, fisetin, butein, icariin and honokiol. The present application also provides a method for preparing the above-mentioned composition. The present application also provides an application of the above-mentioned composition or the product obtained by the above-mentioned preparation method in anti-aging drugs and/or healthcare products. In the technical solution provided by the present application, the nicotinamide mononucleotide can have the function of activating the energy metabolism of an organism and improving the oxidative stress response of the organism, and meanwhile, in synergy with the rutecarpine, the resveratrol, the fisetin, the butein, the icariin and the honokiol, has a good anti-aging effect. And each component in the composition is structurally stable, and will not easily deteriorate and be damaged after preparation of a corresponding product; meanwhile, each component is safe, and has no substantial adverse reactions with the human body. The application of the composition containing nicotinamide mononucleotide provided by the present application in anti-aging drugs/healthcare products solves the technical problem in the prior art for anti-aging products to have good anti-aging effects whilst being harmless to the human body and having stable product quality.

The above are only the preferred embodiments of the present application. It should be pointed out that for those of ordinary skill in the art, without deviating from the principles of the present application, several improvements and modifications can be made, which shall also be considered as the scope of protection of the present application.

1. A composition, wherein the composition comprises the following raw materials: nicotinamide mononucleotide, rutecarpine, resveratrol, fisetin, butein, icariin and honokiol.

2. The composition according to claim 1, wherein the composition comprises the following raw materials in parts by mass: 1-10 parts of nicotinamide mononucleotide, 1-10 parts of rutecarpine, 1-10 parts of resveratrol, 1-10 parts of fisetin, 1-10 parts of butein, 1-10 parts of icariin and 1-10 parts of honokiol.

3. The composition according to claim 2, wherein the composition comprises the following raw materials in parts by mass: 2-8 parts of nicotinamide mononucleotide, 5-7 parts of rutecarpine, 2-5 parts of resveratrol, 3-6 parts of fisetin, 1-5 parts of butein, 6-9 parts of icariin and 2-5 parts of honokiol.

4. The composition according to claim 1, wherein a dosage form of the composition is an oral formulation and/or a parenteral administration dosage form; the oral formulation is selected from any one or more of a tablet, a powder, a capsule, a granule, a pill, a suspension, a syrup, a mixture, a pulvis, and a drop pill; the parenteral administration dosage form is selected from any one or more of an injection, an inhalant, a patch, a suppository and an ointment.

5. A method for preparing the composition according to claim 1, wherein the method comprises the following steps:
step 1, drying and sieving nicotinamide mononucleotide, rutecarpine, resveratrol, fisetin, butein, icariin and honokiol respectively, and then mixing the resulting materials uniformly to obtain a first product;
step 2, drying auxiliary materials and mixing uniformly, and then sieving the resulting materials to obtain a second product;
step 3, dissolving the first product and the second product in water to obtain a mixture and stiffing the mixture for a first time, then heating the mixture and stiffing the mixture for a second time, preparing the resulting materials into a dosage form to obtain the product.

6. The method according to claim 5, wherein, in step 1, a particle size of the sieving is 60-80 meshes; in step 2, a particle size of the sieving is 60-80 meshes.

7. The method according to claim 5, wherein the auxiliary material in step 2 is selected from any one or more of mannitol, microcrystalline cellulose, magnesium stearate, carboxymethyl cellulose, and calcium hydrophosphate.

8. The preparation method according to claim 5, wherein, in step 3, a temperature of the first stiffing is 25-40° C., and a temperature of the second stiffing is 50° C., and a time for the second stiffing is 1 hour.

9. An application of the composition according to claim 1 in anti-aging drugs and/or healthcare products.

10. An application of the product obtained by the method according to claim 5 in anti-aging drugs and/or healthcare products.
